(19) United States
(12) Patent Application Publication
(10) Pub. No.: US 2023/0368929 A1
(43) Pub. Date: Nov. 16, 2023

(54) Collaborative Medical Diagnosis and Treatment System and Method of Use

(71) Applicant: Keleisha Ennis, Jarrettsville, MD (US)

(72) Inventor: Keleisha Ennis, Jarrettsville, MD (US)

(21) Appl. No.: 18/316,678

(22) Filed: May 12, 2023

Related U.S. Application Data
(60) Provisional application No. 63/341,125, filed on May 12, 2022.

(51) Int. Cl. G16H 80/00; G16H 50/20; G16H 40/20; G16H 70/60; G16H 50/70; G16H 10/60

(52) U.S. Cl. CPC G16H 80/00; G16H 50/20; G16H 40/20; G16H 70/60; G16H 50/70; G16H 10/60

(57) ABSTRACT

A system and method for providing medical professionals with an electronic collaborative approach to patient care is disclosed. The system has a recommendation engine and an AI module for selecting one or more diagnoses and associated treatment information from the diagnosis database based on symptoms and other information such as patient information. The system selects one or more professionals for collaboration to discuss and develop treatment plans. In some embodiments, the AI module provides a cut-off value to the stored diagnosis and the cut-off is used for selection or rejection of the diagnosis. The new diagnosis recommended in the collaboration is added to the diagnosis database for future use.

### CROSS-REFERENCE TO RELATED APPLICATION

The present application claims priority to, and the benefit of, U.S. Provisional Application No. 63/341,125, which was filed on May 12, 2022, and is incorporated herein by reference in its entirety.

### FIELD OF THE INVENTION

The present invention relates generally to the field of diagnostic systems. More specifically, the present invention relates to a novel collaborative system for medical professionals for collaborating with each other to discuss cases, symptoms, treatments, and solve unique and puzzling diagnoses. The system provides a website and an application for accessing features of the system allowing professionals to chat and talk to discuss cases and thus, potentially reaching a solution or treatment quicker from insight available from various medical professionals. Accordingly, the present disclosure makes specific reference thereto. Nonetheless, it is to be appreciated that aspects of the present invention are also equally applicable to other like applications, devices, and methods of manufacture.

### BACKGROUND

By way of background, medical professionals such as doctors and nurses are required to stay current with the most recent research and practices in order to give their patients the best care possible. The field of medicine is continually changing and medical experts, even with their significant training and understanding, might struggle to recognize and resolve certain problems. Sometimes a patient may have many underlying medical conditions that complicate treatment or a condition that is unusual or challenging to diagnose. In these circumstances, having additional views and recommendations on a particular ailment or case might aid medical practitioners in providing effective and optimal care for patients. Doctors and nurses can exchange knowledge, discuss treatment choices, and learn new perspectives on a patient's health by working with other medical specialists. Better patient outcomes and a more complete approach to treatment may result from this.

Medical professionals can benefit from hearing another professional's perspective on a certain ailment or circumstance as there may be several different treatment options available for a given issue. Cooperation between medical specialists can also aid in preventing medical errors, which can have detrimental effects on patients. Together, health care providers can identify errors, discuss best practices, and guarantee that patients receive the best treatment possible. Medical professionals desire a system which enables them to collaborate easily and that alleviates long wait times regarding research and can help narrow down solutions related to treatments without delay.

Therefore, there exists a long-felt need in the art for a system that enables medical professionals to obtain insights on different cases and illnesses. There is also a long-felt need in the art for a system that enables doctors, nurse practitioners, physician physicians, nurses, etc. to help other professionals with different treatments and cases with a different perspective or opinion. Additionally, there is a long-felt need in the art for a collaboration system for doctors that improves care for patients with unique and puzzling diagnoses. Moreover, there is a long-felt need in the art for a diagnostic system that enables medical professionals around the world to collaborate to treat patients effectively. Further, there is a long-felt need in the art for a system that uses AI and machine learning, along with medical professionals' advice for providing treatments. Furthermore, there is a long-felt need in the art for a medical system that helps medical professionals to narrow down solutions on treatments without delay. Finally, there is a long-felt need in the art for a differential diagnosis system that enables medical professionals to reach a solution or treatment quicker from insight available from various medical professionals.

The subject matter disclosed and claimed herein, in one embodiment thereof, comprises a system for providing collaboration between medical professionals. The system includes a mobile application or a website accessed in a computing device to access a server system via a communication network, the server system includes an application server or web server, including a recommendation engine module that uses machine learning algorithms for analyzing patient data, symptoms, stored diagnosis information, medical professional information stored in a plurality of databases for providing automatic treatment recommendations and an artificial intelligence (AI) module utilizing natural language processing and deep learning techniques to extract insights from unstructured medical data stored in different databases, and a communication module enabling medical professionals to exchange information about patient cases, including symptoms, treatments, and outcomes, via a chat interface or video conferencing for real-time communication and the ability to share images and documents. The collaboration can be used for making collaborative treatment plans for developing treatment plans for complex or difficult-to-treat conditions.

In this manner, the differential diagnosis system of the present invention accomplishes all of the forgoing objectives and provides users with a software application and a website for accessing a platform that enables medical professionals to collaborate with each other in real-time to solve cases and provided quick and accurate medical treatment to patients. The system also uses AI for automatically or autonomously providing diagnosis to patients. The system improves care for patients with unique and puzzling diagnoses, potentially reaching a solution or treatment quicker from insight available from various medical professionals.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a method for providing collaborative medical diagnosis and treatment among medical professionals. The method further comprising the steps of providing a differential diagnosis system for medical professionals for providing insights into medical cases based on symptoms and to help other professionals with different treatments and cases with a different perspective, using a mobile application or a website accessed in a computing device to access a server system via a communication network, analyzing symptoms, stored diagnosis information, and medical professional information stored in the databases to provide automatic treatment recommendations utilizing machine learning algorithms and natural language processing and deep learning techniques, exchanging information about patient cases, including symptoms, treatments, and outcomes, among medical professionals, and providing visualization tools to help medical professionals identify patterns and insights from symptom data.

In yet another embodiment, a system for providing collaboration between medical professionals is disclosed. The system includes a mobile application or a website accessed in a computing device to access a server system via a communication network, the server system includes an application server or web server, including a recommendation engine module that uses machine learning algorithms for analyzing patient data, symptoms, stored diagnosis information, medical professional information stored in a plurality of databases for providing automatic treatment recommendations and an AI module utilizing natural language processing and deep learning techniques to extract insights from unstructured medical data stored in different databases, and a communication module enabling medical professionals to exchange information about patient cases, including symptoms, treatments, and outcomes, via a chat interface or video conferencing for real-time communication and the ability to share images and documents.

In yet another embodiment, a method for enabling medical professionals to collaborate on developing treatment plans for complex or difficult-to-treat conditions is described. The method includes the steps of selecting one or more diagnosis and treatment information from a diagnosis database based on symptoms and patient information, determining a cut-off value for the selected diagnosis and treatment information, providing the diagnosis as a recommendation if the cut-off is more than a threshold value, automatically or autonomously selecting and notifying one or more medical professionals for collaboration based on their profiles and the query when the cut-off is less than the threshold value, collaboration among the selected professionals for developing one or more treatment plans wherein the one or more treatment plans are added to the diagnosis database for future use.

In yet another embodiment, a platform for medical professionals around the world to provide insight into diagnosis and treatment is disclosed. The platform provides a software application and a website configured to provide chat module for medical professionals to collaborate, the application and the website are further configured to publish blog-like postings containing the diagnosis and treatments. The platform also enables patients to obtain information quicker from medical professionals to complete effective treatment.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of a differential diagnosis system of the present invention in accordance with the disclosed architecture;

FIG. 2 illustrates an exemplary user interface displayed by the application or website for registration of a medical professional in the collaborative medical diagnosis and treatment system of the present invention in accordance with the disclosed architecture;

FIG. 3 illustrates another user interface offered by the collaborative medical diagnosis and treatment system of the present invention for displaying relevant treatment categories to medical professionals in accordance with the disclosed architecture;

FIG. 4 illustrates a flow diagram depicting a process of registration and authentication of a medical professional in the differential diagnosis system of the present invention in accordance with the disclosed architecture;

FIG. 5 illustrates a flow diagram depicting a process of providing automatic diagnosis information and medical professionals recommendation to a requesting medical professional in accordance with the disclosed architecture;

FIG. 6 illustrates a flow diagram depicting a process of receiving a medical request by a medical professional for collaboration in accordance with the disclosed architecture;

FIG. 7 illustrates a flow diagram depicting interaction of a patient with the differential diagnosis system of the present invention for receiving a diagnosis in accordance with the disclosed architecture; and

FIG. 8 illustrates an exemplary user device used for installing the software application of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a system that enables medical professionals to obtain insights on different cases and illnesses. There is also a long-felt need in the art for a system that enables doctors, nurses, etc. to help other professionals with different treatments and cases with a different perspective. Additionally, there is a long-felt need in the art for a collaboration system for doctors that improves care for patients with unique and puzzling diagnoses. Moreover, there is a long-felt need in the art for a diagnostic system that enables medical professionals around the world to collaborate to treat patients effectively. Further, there is a long-felt need in the art for a system that uses AI and machine learning along with medical professionals' advice for providing treatments. Furthermore, there is a long-felt need in the art for a medical system that helps medical professionals to narrow down solutions regarding treatments without delay. Finally, there is a long-felt need in the art for a differential diagnosis system enables medical professionals to reach a solution or treatment quicker from insights available and obtained from various medical professionals.

The present invention, in one exemplary embodiment, is a method for enabling medical professionals to collaborate on developing treatment plans for complex or difficult-to-treat conditions. The method includes the steps of selecting one or more diagnosis and treatment information from a diagnosis database based on symptoms and patient information, determining a cut-off value for the selected diagnosis and treatment information, providing the diagnosis as a recommendation if the cut-off is more than a threshold value, automatically or autonomously selecting and notifying one or more medical professionals for collaboration based on their profiles and the query when the cut-off is less than the threshold value, collaboration among the selected professionals for developing one or more treatment plans wherein the one or more treatment plans are added to the diagnosis database for future use.

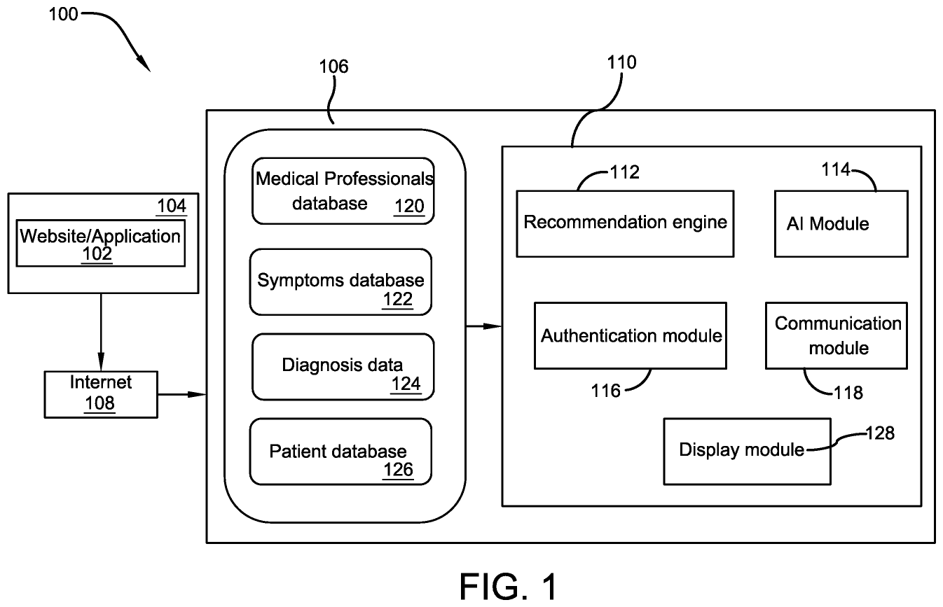

Referring initially to the drawings, FIG. 1 illustrates a schematic view of differential diagnosis system of the present invention in accordance with the disclosed architecture. The differential diagnosis system 100 of the present embodiment is designed as a platform for medical professionals to provide insights into medical cases based on symptoms and allow them to help other professionals with different treatments and cases with a different perspective, allowing for a wider range of treatment options and a broader perspective on medical cases. More specifically, the system 100 uses a mobile application or a website 102 accessed in a computing device 104 such as a smartphone or any other electronic computing device. A server system 106 is configured to be accessed using a communication network 108 such as Internet by a user using the application or website 102 for using functionalities provided by the system 100.

The server system 106 is designed to be scalable and capable of handling a high load of concurrent users. An application server or web server 110 includes a recommendation engine 112 module that uses machine learning algorithms for analyzing patient data, symptoms, stored diagnosis information, medical professional information stored in the databases as described later in the disclosure for providing automatic treatment recommendations. The AI module 114 utilizes natural language processing and deep learning techniques to extract insights from unstructured medical data, such as medical literature and patient notes stored in different databases.

The authentication module 116 is configured to authenticate medical professionals and patients before allowing access of the data stored in the system 100 and as a result, ensures secure access to the system 100. Further, the authentication module 116 also provides access based on role and permissions and therefore, provide a layered security level to the system 100. The communication module 118 enables medical professionals to exchange information about patient cases, including symptoms, treatments, and outcomes. The module 118 provides a plurality of means, including, but not limited to, chat interface, video conferencing for real-time communication and the ability to share images and documents among medical professionals. A display module 128 is configured to provide visualization tools to help medical professionals identify patterns and insights from symptoms data. Further, the display module 128 is also configured to display the information retrieved from one or more databased in different formats, including, but not limited to HTML, XML, JavaScript, and any other format known in the art.

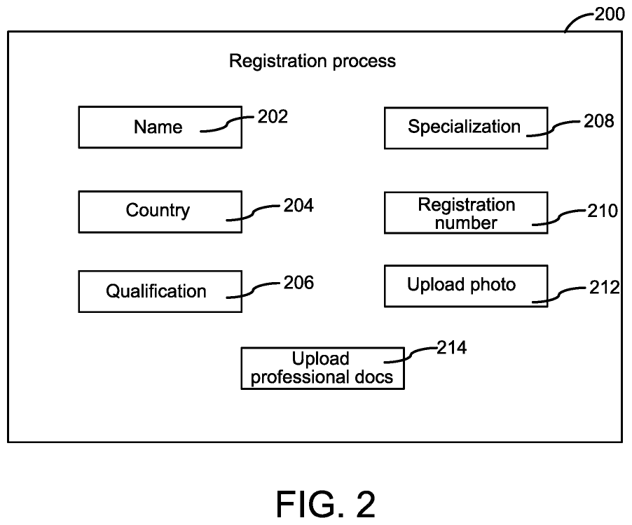

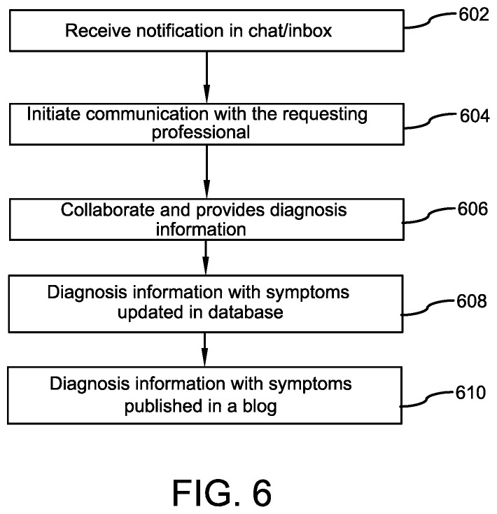

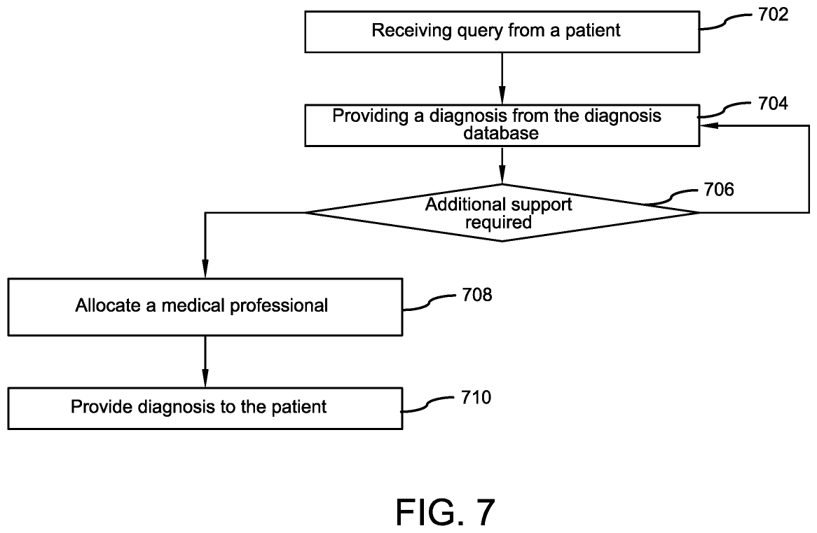

A medical professional database 120 is configured to contain information about registered medical professionals, including their personal and/or professional information registered during registration process as illustrated in FIG. 2, specialties, location, experience, credentials and more. The information stored in the medical professional database 120 is used for automatically or autonomously selecting a medical professional for providing insights and point of view for medication and treatment as described in FIGS. 6 and 7. The ailment symptoms database 122 stores information about various symptoms and their associated ailments. The database 122 is updated periodically by the recommendation engine 112 and the AI module 114 by including new symptoms and associated ailments.

The diagnosis database 124 stores information about past diagnoses and treatments for various ailments. The stored diagnosis are learnt by the AI module 114 and a cut-off score is provided to each diagnosis and/or treatment for a set of symptoms and ailments. The diagnosis that are above a threshold cut-off are automatically or autonomously selected by the recommendation engine 112 as described in FIG. 6. The diagnosis database 124 can include information which can be one or all of the medicines, surgery, physical therapy, rest, diagnostic testing, and more. The patient database 126 contains information about individual patient cases, including their symptoms, medical history, and treatment options and can be referred by medical professionals registered in the system 100 for providing additional support as described in FIG. 7.

The recommendation module 112 using the machine learning algorithms can identify and match similar cases based on symptoms and medical history. This can allow medical professionals to quickly identify relevant cases and potential diagnoses and provide more targeted insights and treatment recommendations.

It should be noted that the information stored in different databases of the system 100 comply with all relevant laws and regulations, including those related to patient privacy and medical confidentiality. The system 100 is also designed to be easily configurable to meet the specific needs of different healthcare organizations and medical specialties. The system 100 of the present invention provides the proper virtual connectivity between the medical professionals and with the patients as well. The professionals can help, collaborate, instruct, and recommend performing the necessary medication and diagnosis through textual, image, audio and/or visual means.

FIG. 2 illustrates an exemplary user interface displayed by the application or website for registration of a medical professional in the collaborative medical diagnosis and treatment system of the present invention in accordance with the disclosed architecture. The registration user interface 200 is designed to be easy to use and efficient, ensuring that all necessary information is captured accurately and in a timely manner for registration of a medical professional. The interface 200 provides a name input box 202 for inputting name, a country input box 204 for inputting country, a medical qualification input box 206 for inputting the qualification degrees, a medical specialization input box 208 for inputting specialization such as cardiology, pediatrics, neurosurgery and more. The interface 200 also requires valid registration number 210 of the medical professional for authentication purposes. For creating a profile of the medical professional, a photo is uploaded using the upload photo box 212 and for verifying the credentials of medical professionals, professional documents are uploaded using the upload professional docs box 214.

The registration user interface 200 is designed to be customizable to meet the needs of different regulatory bodies and jurisdictions. The input boxes can be modified to capture additional information as required, such as license number, qualifications, and experience. In operation, a medical professional enters the required information into the input boxes, along with any additional information required by the system 100 and relevant regulatory body. The registration information is stored in medical professional database 120 after verification of the professional's medical credentials and medical qualifications.

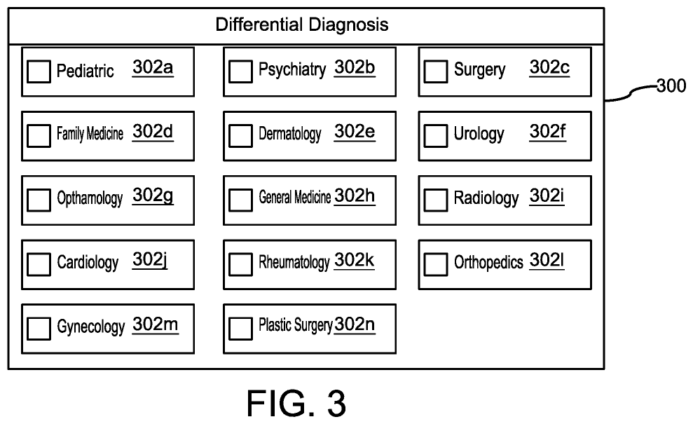

FIG. 3 illustrates another user interface offered by the collaborative medical diagnosis and treatment system of the present invention for displaying relevant treatment categories to medical professionals in accordance with the disclosed architecture. The user interface 300 displays a list of treatment or medical categories 302a-n for which a medical professional can seek advice or can give recommendations. A medical professional upon successful registration can select one or more of the categories 302a-n as per the specialization or preferences. Alternatively, the recommendation engine 112 of the server system 106 automatically or autonomously displays a list of categories based on the experience, qualification, specialization and more submitted during registration process by the medical professional. The categories interface 200 is designed to be customizable to include and exclude any new types of categories. A medical professional can select a category to access the store of diagnoses, treatments, symptoms for insights and broad point of view.

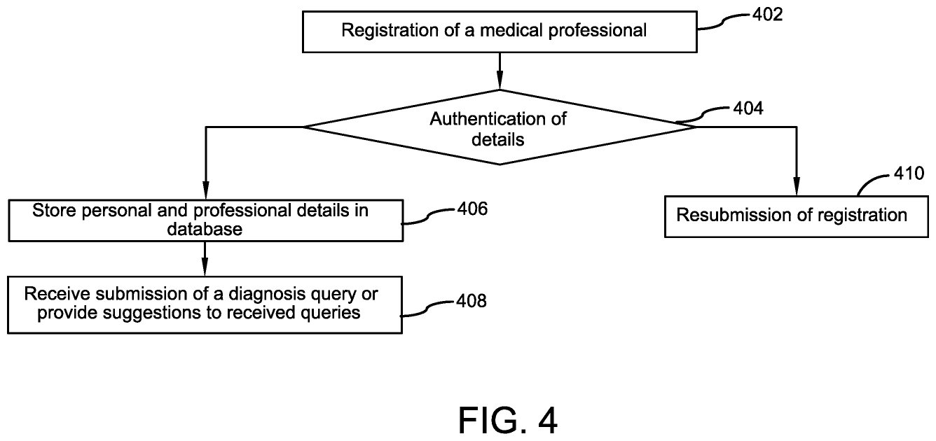

FIG. 4 illustrates a flow diagram depicting a process of registration and authentication of a medical professional in the differential diagnosis system of the present invention in accordance with the disclosed architecture. Initially, using the interface 200 as illustrated in FIG. 2, registration information of a professional is received by the application server 110 (Step 402). Then, authentication of the registration information is performed by the authentication module (Step 404). The authentication module 116 can contact 3rd party databases such as regulatory authorities' databases for verification of the registration information submitted by a medical professional. If the authentication module successfully authenticates the registration information, then, the registration information is stored in the secure and encrypted form in the medical professional database (Step 406). After successful authentication and registration, a medical professional starts to submit queries to collaborate with other medical professionals registered in the system (Step 408). If the authentication of registration information has failed, then, the process moves to step 410 where the medical professional is asked to resubmit the registration information for authentication.

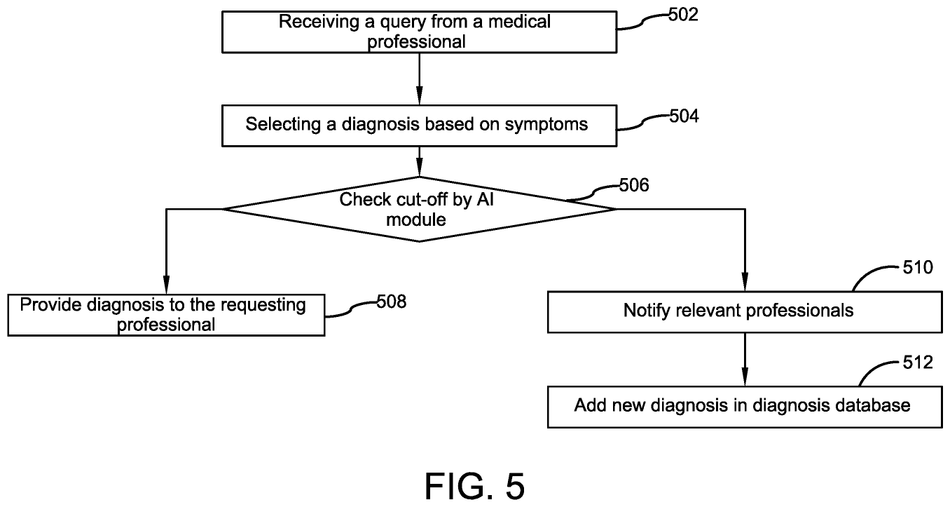

FIG. 5 illustrates a flow diagram depicting a process of providing automatic diagnosis information and medical professionals recommendation to a requesting medical professional in accordance with the disclosed architecture. Initially, a query is received from a medical professional wherein the query can be for recommendations for treatment or diagnosis based on symptoms and the request can include symptoms, patient information, past diagnosis, and any other information based on preferences and requirements of the medical professional (Step 502). Then, based on the received information, the recommendation engine and the AI module together select one or more diagnosis and treatment information from the diagnosis database (Step 504). Thereafter, the AI module checks the cut-off value for the selected diagnosis and treatment information (Step 506). If the cut-off value is more than a threshold value, then, the diagnosis is provided to the requesting professional as a recommendation (Step 508).

If the cut-off value for the selected diagnosis and treatment information is less than the threshold value, then, the recommendation engine based on profiles of the registered professionals and query, automatically or autonomously selects, and notifies one or more medical professionals for collaboration (Step 510). The new diagnosis recommended in the collaboration can be added in the diagnosis database along for future use (Step 512). In some embodiments, the requesting user can manually select one or more medical professionals for collaboration.

In addition to providing insights and recommendations on specific cases, the medical professionals using the system 100 can collaborate on developing treatment plans for complex or difficult-to-treat conditions. This enables a more comprehensive and collaborative approach to patient care, with multiple experts providing input and feedback on treatment options.

FIG. 6 illustrates a flow diagram depicting a process of receiving a medical request by a medical professional for collaboration in accordance with the disclosed architecture. Initially, a registered medical professional receives a notification for collaboration in the integrated chat box or any other means of communication from other medical professionals (Step 602). Then, the medical professional initiates communication with the requesting user using the integrated communication means (Step 604). The communication can be done by any audio, textual, video, augmented reality, and virtual reality means. Thereafter, based on the symptoms and expertise and specialization of the medical professional, diagnosis and treatment are provided to the requesting user (Step 606). Then, the diagnosis information is updated in one or more databases of the system 100 and in some cases, a notification can be automatically or autonomously transmitted to all medical professionals of the same specialization (Step 608). Finally, the information is also published automatically or autonomously in a medical blog and is published in one or more webpages published the server system 106 (Step 610).

FIG. 7 illustrates a flow diagram depicting interaction of a patient with the differential diagnosis system 100 of the present invention for receiving a diagnosis in accordance with the disclosed architecture. Initially, a query from a registered patient is received by the system 100 (Step 702). The query can include personal details of the patient as well along with any medical history. The patient can register using a similar process of FIG. 2. Then, the system 100 automatically or autonomously provides a diagnosis from the diagnosis database (Step 704). This step provides the advantage that a patient receives instant diagnosis from the system 100 which can be extremely useful in emergency medical conditions as well. If additional support is requested by the patient as determined in step 706, then, the system in step 708, allocates a registered medical professional automatically or autonomously to the patient. The registered professional automatically or autonomously receives all the medical history and other details of the patient for facilitating a quick treatment and recommendation. The selected professional can communicate with the patient and provide diagnosis (Step 710). It should be appreciated that the selected professional can collaborate with other professional as described in FIGS. 5 and 6.

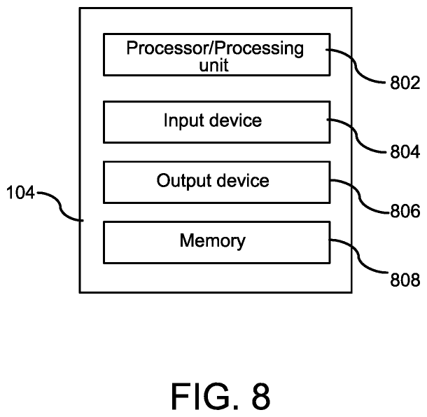

FIG. 8 illustrates an exemplary user device 104 used for installing the software application 102 of the present invention in accordance with the disclosed architecture. The processing unit 802 may include suitable logic, instructions, circuitry, interfaces, and/or codes for executing various operations, such as the operations associated with the user device 104, or the like. The processing unit 802 may be configured to control one or more operations executed by the user device 104 in response to the input received at the user device 104 from the user. The processor 802 executes the computer readable instructions stored in the application 102. Examples of the processing unit 802 may include, but are not limited to, an application-specific integrated circuit (ASIC) processor, a reduced instruction set computing (RISC) processor, a complex instruction set computing (CISC) processor, a field-programmable gate array (FPGA), a Programmable Logic Control unit (PLC), and the like. Embodiments of the present disclosure are intended to include or otherwise cover any type of the processing unit 802 including known, related art, and/or later-developed processing units. The user device 104 can further include one or more computer executable applications configured to be executed by the processing unit 802. The one or more computer executable applications may include suitable logic, instructions, and/or codes for executing various operations. The one or more computer executable applications may be stored in the memory 808. The one or more computer executable applications includes the application 102.

The user device 104 includes input device(s) 804 such as a touch input device, voice input device, etc. for entering data and information. Preferably, the touch interface of the user device 104 is used as the input and various buttons/tabs shown on the application 102 are pressed or clicked by the user. Other input devices such as camera and microphone are used during video chatting by the user. The display of the user device 104 also acts as the output device 806 for displaying various contents (i.e., text, images, videos, icons, and/or symbols, etc.) to the user. The display can include a touch screen, and may receive, for example, a touch, gesture, proximity, or hovering input using an electronic pen or a part of a user's body.

Electronic device 104 has memory 808 used for storing programs (sequences of instructions) or data (e.g., program state information) on a temporary or permanent basis for use in the computer system. Memory 808 can be configured for short-term storage of information as volatile memory and therefore not retain stored contents if powered off. Examples of volatile memories include random access memories (RAM), dynamic random-access memories (DRAM), static random-access memories (SRAM), and other forms of volatile memories known in the art. The processor 802, in combination with one or more of memory 808, input device(s) 804, output device(s) 806 can be utilized to provide users the ability to execute instructions on the application 102.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “differential diagnosis system”, “collaborative medical diagnosis and treatment system”, “collaborative medical diagnosis and treatment platform with AI and recommendation engine”, and “system” are interchangeable and refer to the collaborative medical diagnosis and treatment system 100 of the present invention.

Notwithstanding the forgoing, the collaborative medical diagnosis and treatment system 100 of the present invention can be of any suitable size and configuration as is known in the art without affecting the overall concept of the invention, provided that it accomplishes the above-stated objectives. One of ordinary skill in the art will appreciate that the collaborative medical diagnosis and treatment system 100 as shown in the FIGS. are for illustrative purposes only, and that many other configurations of the collaborative medical diagnosis and treatment system 100 are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An electronic medical diagnosis system for connecting medical professionals, the electronic medical diagnosis system comprising:
an electronic platform including a computing device, a communication network, and an application server;
a server system configured to be accessed using said communication network;
wherein said communication network is an Internet;
wherein said application server having a recommendation engine module including machine learning algorithms for storing and analyzing patient data, symptoms, stored diagnosis information, and medical professional information;
wherein said storing including an electronic database;
an authentication module configured to authenticate medical professionals and patients before allowing access of said data stored in said server system;
wherein said authentication module provides secure access to said server system;
wherein said authentication module further providing selective access based on a role and a permission;
a communication module for medical professionals to exchange information about said patient data, said symptoms, said stored diagnosis information, medical treatments, and medical outcomes;
wherein said communication module includes communication means between said medical professionals selected from a group consisting of a chat interface, a video conferencing, an image sharing, and a document sharing;
a display module for providing visualization tools to said medical professionals for identifying patterns and insights from said symptoms;
wherein said display module configured to display said image sharing and said document sharing in a format selected from a group consisting of an HTML, an XML, and a JavaScript; and
an AI module having natural language processing for analyzing and extracting future said medical diagnoses from past said medical diagnoses.

2. The electronic medical diagnosis system for connecting medical professionals of claim 1 further comprising a medical professional database configured to contain information about said medical professionals and professional information registered during a registration process.

3. The electronic medical diagnosis system for connecting medical professionals of claim 2, wherein said professional information includes information selected from a specialty, a location, an experience, and a credential.

4. The electronic medical diagnosis system for connecting medical professionals of claim 3, wherein said medical professional database autonomously selects said medical professional for providing said insights for said medication and said treatment.

5. The electronic medical diagnosis system for connecting medical professionals of claim 4, wherein said insights from unstructured medical data, such as medical literature and patient notes stored in different databases.

6. The electronic medical diagnosis system for connecting medical professionals of claim 5 further comprising an ailment symptoms database storing information about various said symptoms and associated ailments.

7. The electronic medical diagnosis system for connecting medical professionals of claim 6, wherein said aliment symptoms database is updated periodically by said recommendation engine and said AI module by including new symptoms and new associated ailments.

8. The electronic medical diagnosis system for connecting medical professionals of claim 7, wherein said stored diagnosis information is a diagnosis database storing information about past diagnoses and past treatments for various said ailments.

9. The electronic medical diagnosis system for connecting medical professionals of claim 8, wherein said application is a website.

10. The electronic medical diagnosis system for connecting medical professionals of claim 9, wherein said computing device is a smartphone.

11. The electronic medical diagnosis system for connecting medical professionals of claim 10, wherein said diagnosis database includes diagnoses selected from a group consisting of a medicine, a surgery, a physical therapy, a rest, and a diagnostic testing.

12. The electronic medical diagnosis system for connecting medical professionals of claim 11 further comprising a patient database including said patient data selected from a group consisting of a patient case, said symptoms, a medical history, and a treatment option.

13. The electronic medical diagnosis system for connecting medical professionals of claim 12 further comprising a recommendation module using the machine learning said AI module algorithms for identifying and matching similar said patient cases based on said symptoms and said medical history.

14. The electronic medical diagnosis system for connecting medical professionals of claim 13, wherein said authentication module further including a profile of said medical professional including information selected from a group consisting of a medical qualification, a medical credential, a medical specialization, a registration number, a photo, and a professional document.

15. The electronic medical diagnosis system for connecting medical professionals of claim 14 further comprising a user interface having a display of treatment categories for accessing by said medical professional to selectively seek advice or provide recommendations.

16. The electronic medical diagnosis system for connecting medical professionals of claim 15, wherein said treatment categories are autonomously displayed based on said experience and said qualification of said medical professional submitted during said registration.

17. A method for providing medical professionals with an electronic collaborative approach to patient care, the method comprising the steps of:
providing an electronic platform including a computing device, a communication network, and an application server;
accessing a server system using said communication network, wherein said communication network is an Internet;
storing and analyzing patient data, symptoms, stored diagnosis information, and medical professional information, wherein said application server having a recommendation engine module including machine learning algorithms for said storing and said analyzing and further wherein said storing including an electronic database;
authenticating medical professionals and patients using an authentication module before allowing access of said data stored in said server system, wherein said authentication module provides secure access to said server system;
providing selective access to said server system based on a role and a permission;
providing a communication module for medical professionals to exchange information about said patient data, said symptoms, said stored diagnosis information, medical treatments, and medical outcomes, wherein said communication module includes communication means between said medical professionals selected from a group consisting of a chat interface, a video conferencing, an image sharing, and a document sharing;
providing visualization tools to said medical professionals with a display module;
identifying patterns and insights from said symptoms, wherein said display module configured to display said image sharing and said document sharing in a format selected from a group consisting of an HTML, an XML, and a JavaScript; and
analyzing and extracting future said medical diagnoses from past said medical diagnoses using an AI module having natural language processing.

18. A method for providing medical professionals with an electronic collaborative approach to patient care, the method comprising the steps of:
providing an electronic platform including a computing device, a communication network, and an application server;
accessing a server system using said communication network;
storing and analyzing patient data, symptoms, stored diagnosis information, and medical professional information, wherein said application server having a recommendation engine module including machine learning algorithms for said storing and said analyzing and further wherein said storing including an electronic database;
authenticating medical professionals and patients using an authentication module before allowing access of said data stored in said server system, wherein said authentication module provides secure access to said server system;
providing selective access to said server system based on a role and a permission;
providing a communication module for medical professionals to exchange information about said patient data, said symptoms, said stored diagnosis information, medical treatments, and medical outcomes, wherein said communication module includes communication means between said medical professionals selected from a group consisting of a chat interface, a video conferencing, an image sharing, and a document sharing;
providing visualization tools to said medical professionals with a display module;
identifying patterns and insights from said symptoms, wherein said display module configured to display said image sharing and said document sharing in a format selected from a group consisting of an HTML, an XML, and a JavaScript;
analyzing and extracting future said medical diagnoses from past said medical diagnoses using an AI module having natural language processing; and
further comprising an ailment symptoms database storing information about various said symptoms and associated ailments, wherein said aliment symptoms database is updated periodically by said recommendation engine and said AI module by including new symptoms and new associated ailments, and further wherein said stored diagnosis information is a diagnosis database storing information about past diagnoses and past treatments for various said ailments.

19. The method of electronic collaborative approach to patient care of claim 18 further comprising the step of registering said medical professionals in a medical professional database configured to contain information about said medical professionals.

20. The method of electronic collaborative approach to patient care of claim 19, wherein said professional information includes information selected from a specialty, a location, an experience, and a credential, and further wherein said medical professional database autonomously selects said medical professional for providing said insights for said medication and said treatment.
